(19) United States
(12) Patent Application Publication
(10) Pub. No.: US 2010/0082109 A1
(43) Pub. Date: Apr. 1, 2010

(54) EXPANDABLE INTERVERTEBRAL IMPLANT

(75) Inventors: E. Skott GREENHALGH, Lower Gwynedd, PA (US); John-Paul ROMANO, Chalfont, PA (US)

Correspondence Address: LEVINE BAGADE HAN LLP, 2400 GENG ROAD, SUITE 120, PALO ALTO, CA, 94303, US

(73) Assignee: Stout Medical Group, L.P., Perkasie, PA (US)

(21) Appl. No.: 12/564,616

(22) Filed: Sep. 22, 2009

Related U.S. Application Data
(60) Provisional application No. 61/099,156, filed on Sep. 22, 2008.

(51) Int. Cl. A61F 2/44

(52) U.S. Cl. 623/17.15

(57) ABSTRACT

An expandable intervertebral implant is disclosed. The implant can include first and second members capable of being expanded upon movement of first and second wedges. The first and second wedges, while being capable of moving with respect to each other and the first and second members can also be attached to the first and second members. In addition, the first and second wedges can be capable of moving only in a first direction, while movement in a second direction can be inhibited. The first and second wedges can also be prevented from torsionally moving with respect to the first and second members.

### BACKGROUND OF THE INVENTION

Surgeons are performing more and more spinal surgeries to correct different spinal defects in the hopes of reducing pain and restoring normal or close to normal movement. One area of particular interest lies in the restoration of normal spacing between adjacent vertebral bodies. Whether due to the degeneration of the intervertebral disc over time or because of an injury, a decrease in spacing between vertebral bodies can cause a myriad of problems for a patient, the least of which is pain resulting from the pinching of nerves between the bodies. Correcting this problem is often very important to returning a patient to his or her normal level of activity and/or managing the pain associated with a degenerative spinal problem.

Over the years, there have been many different techniques employed in restoring the normal disc space. For instance, solid fusion devices have been implanted in many patients in the hopes of both restoring normal disc spacing and preventing further degeneration of the space by fusing the vertebral bodies to one another. Recently, there has been a trend to both restore the disc spacing and allow natural movement of the adjacent vertebral bodies with respect to one another. Nonetheless, there exist certain extreme cases of degradation of the disc space which require extreme measures in order to restore the natural spacing.

Often, the decrease in spacing will be so drastic that some amount of distraction of the adjacent vertebral bodies will be required. Although this distraction is sometimes achieved through the use of various tools, the desire for faster and more efficient surgical techniques favors the elimination of superfluous surgical steps. Thus, there exists a need for an intervertebral implant which is implantable in an unexpanded state and easily expandable to restore the disc space, thereby negating the need for additional tools and the additional surgical steps of using them.

### SUMMARY OF THE INVENTION

An expandable implant for implantation between two vertebral bodies is disclosed. The implant can include a first member, the first member including a first vertebral contact surface and a first interior surface, a second member, the second member including a second vertebral contact surface and a second interior surface, the first and second interior surfaces facing towards one another, a strut attached to both the first and second members, and a wedge disposed between the first and second interior surfaces and attached to at least one of the first or second members. Movement of the wedge in a first direction can cause movement of at least one of the first or second members in a second direction.

The wedge can be attached to at least one of the first or second members by a deformable tether. The implant can include first and second wedges, where movement of the first and second wedges towards one another causes an increase in a distance between the first and second interior surfaces. The first and second wedges can each be attached to both of the first and second members by a deformable tether. One of the first or second wedges can include a bulleted or rounded surface for aiding in insertion of the expandable implant between the two vertebral bodies. The first wedge can include first and second angled wedge surfaces for cooperating with first and second angled interior surfaces of the first and second members, respectively. The second wedge can include third and fourth angled wedge surfaces for cooperating with third and fourth angled interior surfaces of the first and second members, respectively. Movement of the first and second wedges towards one another can be permitted, while movement of the first and second wedges away from one another can be prevented. This can be the case because the first, second, third, and fourth wedge surfaces and the first, second, third, and fourth interior surfaces can each include teeth. The first and second members and the first and second wedges can also cooperate to define at least one aperture through the implant adapted for bone growth therethrough.

Another expandable implant for implantation between two vertebral bodies is disclosed. The implant can include a first member, the first member including a first vertebral contact surface and a first interior surface, a second member, the second member including a second vertebral contact surface and a second interior surface, the first and second interior surfaces facing towards one another, a strut attached to both the first and second members, and first and second wedges disposed between the first and second interior surfaces, one of the first or second wedges including a bulleted or rounded surface for aiding in insertion of the expandable implant between the two vertebral bodies. Movement of the first wedge towards the second wedge can causes an increase in a distance between the first and second interior surfaces.

Each of the first and second wedges can be attached to each of the first and second members by deformable tethers. The first wedge can include first and second angled wedge surfaces for cooperating with first and second angled interior surfaces of the first and second members, respectively. The second wedge can include third and fourth angled wedge surfaces for cooperating with third and fourth angled interior surfaces of the first and second members, respectively. Movement of the first and second wedges towards one another can be permitted, while movement of the first and second wedges away from one another can be prevented. This can be the case because the first, second, third, and fourth wedge surfaces and the first, second, third, and fourth interior surfaces each include teeth. The first and second members and the first and second wedges can cooperate to define at least one aperture through the implant adapted for bone growth therethrough.

An expandable implant for implantation between two vertebral bodies is disclosed. The implant can include a first member. The first member can include a first vertebral contact surface and a first interior surface, a second member, the second member including a second vertebral contact surface and a second interior surface, the first and second interior surfaces facing towards one another, a strut attached to both the first and second members, and first and second wedges disposed between the first and second interior surfaces. Movement of the first wedge towards the second wedge can cause an increase in a distance between the first and second interior surfaces, and at least one of the first and second wedges can be prevented from torsionally moving with respect to the first and second members.

Each of the first and second wedges can be attached to each of the first and second members by deformable tethers. The first wedge can include first and second angled wedge surfaces for cooperating with first and second angled interior surfaces of the first and second members, respectively. The second wedge can include third and fourth angled wedge surfaces for cooperating with third and fourth angled interior surfaces of the first and second members, respectively. Movement of the first and second wedges towards one another can be permitted, while movement of the first and second wedges away from one another can be prevented. This can be the case because the first, second, third, and fourth wedge surfaces and the first, second, third, and fourth interior surfaces can each include teeth. The first and second members and the first and second wedges can cooperate to define at least one aperture through the implant adapted for bone growth therethrough. The first and second members can include either a depression or a protuberance, and the first and second wedges can include the other of a depression or a protuberance. The first and second members can include a tongue, a pin, or an elongate projection, and the first and second wedges can include either a groove or a channel.

Yet another expandable implant for implantation between two vertebral bodies is disclosed. The implant can include a first member, the first member including a first vertebral contact surface and a first interior surface having a first and third angled interior surfaces, a second member, the second member including a second vertebral contact surface and a second interior surface having second and fourth angled interior surfaces, the first and second interior surfaces facing towards one another, a strut attached to both the first and second members, a first wedge disposed between the first and second interior surfaces, the first wedge including first and second angled wedge surfaces for cooperating with the first and second angled interior surfaces of the first and second members respectively, and a second wedge disposed between the first and second interior surfaces, the second wedge including third and fourth angled wedge surfaces for cooperating with the third and fourth angled interior surface of the first and second members respectively. Movement of the first wedge towards the second wedge causes an increase in a distance between the first and second interior surfaces, and movement of the first and second wedges towards one another can be permitted, while movement of the first and second wedges away from one another can be prevented.

The first, second, third, and fourth wedge surfaces and the first, second, third, and fourth interior surfaces can each include teeth. The first and second members and the first and second wedges can cooperate to define at least one aperture through the implant adapted for bone growth therethrough.

Yet another expandable implant for implantation between two vertebral bodies is disclosed. The implant can include a first member, the first member including a first vertebral contact surface and a first interior surface having a first and third angled interior surfaces, a second member, the second member including a second vertebral contact surface and a second interior surface having second and fourth angled interior surfaces, the first and second interior surfaces facing towards one another, a plurality of struts attached to both the first and second members, a first wedge disposed between the first and second interior surfaces, the first wedge including first and second angled wedge surfaces for cooperating with the first and second angled interior surfaces of the first and second members respectively, a first tether connecting the first wedge to one of the first or second members, a second wedge disposed between the first and second interior surfaces, the second wedge including third and fourth angled wedge surfaces for cooperating with the third and fourth angled interior surface of the first and second members respectively, and a first tether connecting the first wedge to one of the first or second members. Movement of the first wedge towards the second wedge causes an increase in a distance between the first and second interior surfaces, and the first, second, third, and fourth wedge surfaces and the first, second, third, and fourth interior surfaces each include teeth. One of the first or second wedges can include a bulleted or rounded surface for aiding in insertion of the expandable implant between the two vertebral bodies.

A method of implanting an expandable implant between two vertebral bodies is disclosed. The method can include the steps of inserting the expandable implant between two vertebral bodies. The implant can have a first member, a second member, and a wedge disposed between the first and second members and attached to at least one of the first or second members. The method also includes the step of moving the wedge in a first direction so as to cause movement of the first and second members which in turn causes movement of the vertebral bodies away from one another.

The implant can further include at least one deformable strut and more than one wedge. Each wedge can be attached to at least one of the first or second members by a deformable tether. In some cases, the wedges can be attached to both members by deformable tethers. The implant can further include structure which allows for the movement of the at least one wedge in a first direction, but prevents movement of the wedge in an opposition direction. The wedge can be prevented from torsionally rotating with respect to the first and second members.

### BRIEF DESCRIPTION OF THE DRAWINGS

A more complete appreciation of the subject matter of the disclosure and the various advantages thereof can be realized by reference to the following detailed description in which reference is made to the accompanying drawings in which:

FIG. 1 is a front perspective view of a variation of an expandable intervertebral implant in a generally unexpanded state.

FIG. 2 is a rear perspective view of the expandable intervertebral implant shown in FIG. 1.

FIG. 3 is a side perspective view of the expandable intervertebral implant shown in FIG. 1.

FIG. 4 is a top view of the expandable intervertebral implant shown in FIG. 1.

FIG. 5 is a side view of the expandable intervertebral implant shown in FIG. 1.

FIG. 6 is a front perspective view of the expandable intervertebral implant shown in FIG. 1 in a fully expanded state.

FIG. 7 is a perspective view of a variation of the expandable intervertebral implant.

FIG. 8 is another perspective view of the expandable intervertebral implant shown in FIG. 7.

FIG. 9 is a side view of the expandable intervertebral implant shown in FIG. 7 in a fully expanded state.

FIG. 10 is a perspective view of a variation of the expandable intervertebral implant.

FIG. 11 is another perspective view of the expandable intervertebral implant shown in FIG. 10.

FIG. 12 is an enlarged view of a portion of the expandable intervertebral implant shown in FIG. 10.

### DETAILED DESCRIPTION

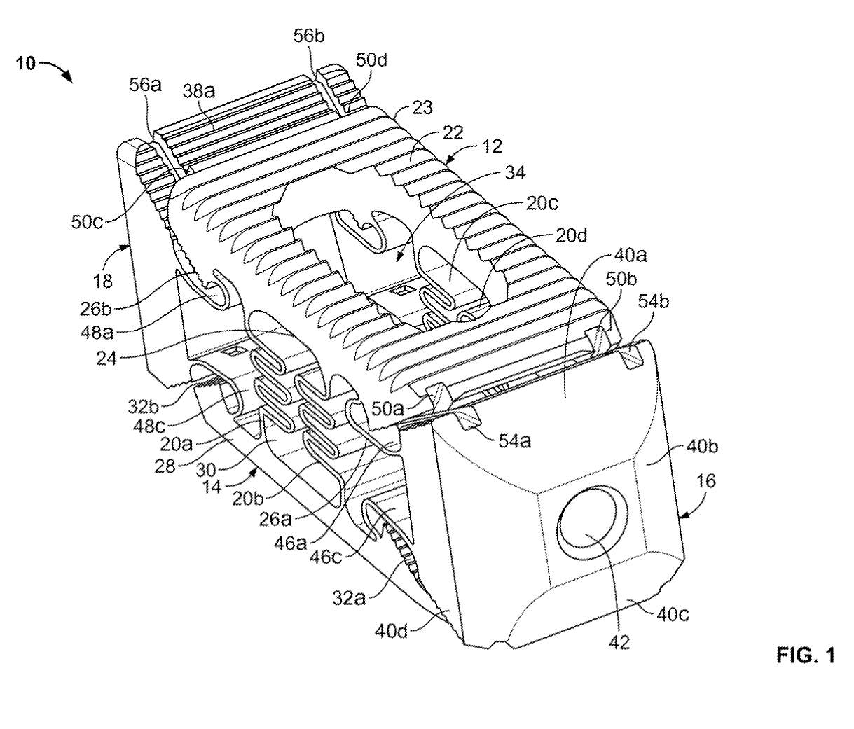

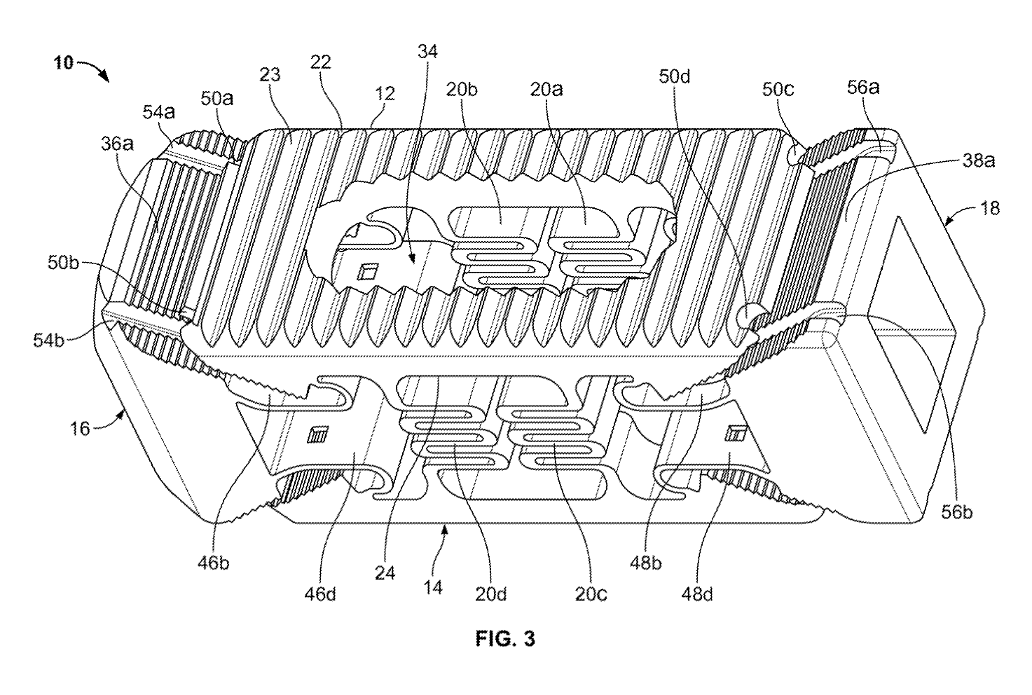

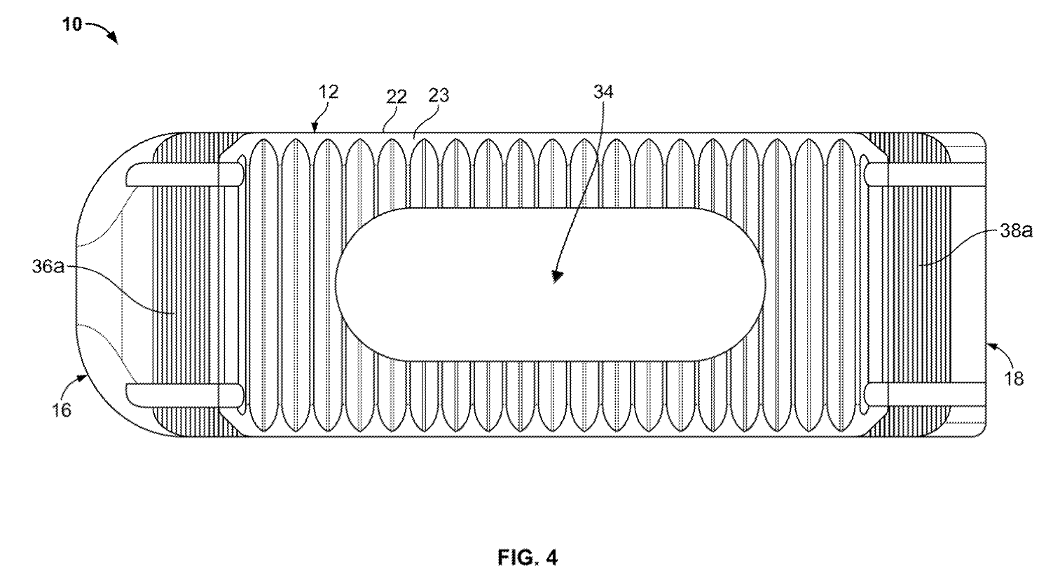

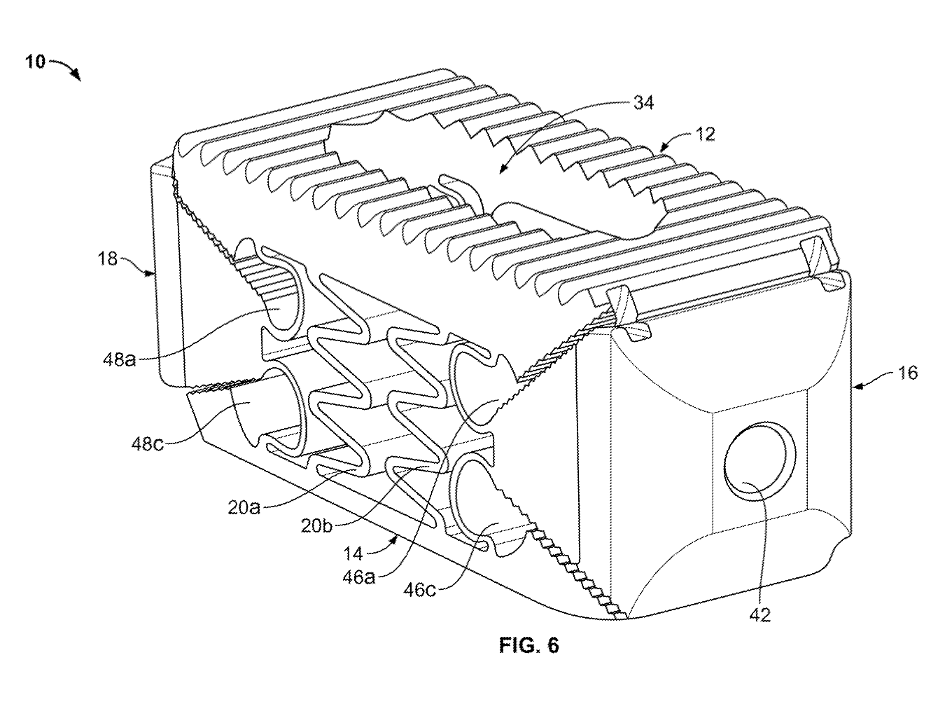

Referring to the drawings, wherein like reference numerals refer to like elements, FIGS. 1-6 depict a first variation expandable intervertebral implant, designated generally by reference numeral 10. As is shown in the drawings, implant 10 includes, among other elements that will be discussed below, a first member 12, a second member 14, a first wedge 16, a second wedge 18, and a plurality of struts 20a-d. Implant 10 is designed so that is capable of expanding from a generally unexpanded state (shown in FIGS. 1-5) to a fully expanded state (shown in FIG. 6), as well as several different partial expended states therebetween. The specific details of the structure and the operation of implant 10 will be discussed further below.

As is shown in FIGS. 1-6, first and second members 12 and 14 are generally planar plate-like elements capable of contacting and supporting a portion of vertebral bodies implant 10 is inserted between. First member 12 includes a first vertebral body contacting surface 22 and a first interior surface 24 having two first angled interior surfaces 26a and 26b. Likewise, second member includes a second vertebral body contacting surface 28 and a second interior surface 30 having two second angled interior surfaces 32a and 32b. First and second vertebral body contacting surfaces 22 and 28 can include bone engaging elements. For example, as is shown in FIGS. 1-6, first vertebral body contacting surface 22 includes projections 23 and second vertebral body contacting surface 28 includes projections 29. These projections are capable of biting into a portion of the bone of the adjacent vertebral bodies implant 10 is inserted between. First angled interior surfaces 26a and 26b can include teeth 27a and 27b, respectively, while second angled interior surfaces 32a and 32b can include teeth 33a and 33b, respectively. First member 12 can define a first aperture 34 and second member 14 can define a second aperture 35 (only partially shown).

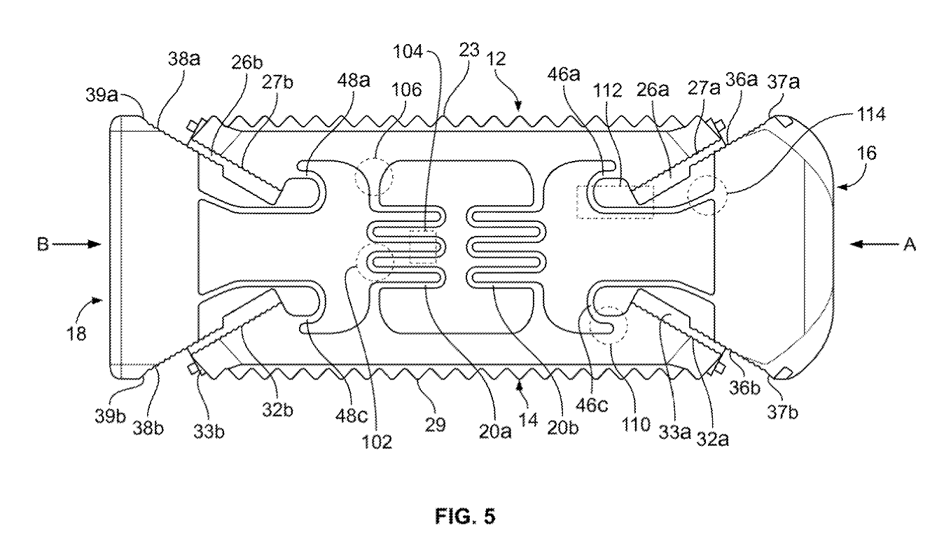

As is also shown in FIGS. 1-6, first and second wedges 16 and 18 are somewhat triangular and include surfaces capable of cooperating with the above-discussed first and second angled interior surfaces. Specifically, first wedge 16 includes first and second angled wedge surfaces 36a and 36b for cooperation with first angled interior surface 26a and second angled interior surface 32a, and second wedge 18 includes third and fourth angled wedge surfaces 38a and 38b for cooperation with first angled interior surface 26b and second angled interior surface 32b. The various wedge surfaces can include similar teeth to those discussed above in connection with first and second angled interior surfaces. For instance, as is shown in FIG. 5, first and second angled wedge surfaces 36a and 36b include teeth 37a and 37b, respectively, and third and fourth angled wedges surfaces 38a and 38b include teeth 39a and 39b, respectively. The different cooperating teeth (i.e., 27a and 37a, 27b and 39a, 33a and 37b, and 33b and 39b) can allow for movement of first and second wedges 16 and 18 with respect to first and second members 12 and 14 in one direction, but prevent it in an opposite direction. The wedges can exhibit any shape suitable for use in expansion of implant 10.

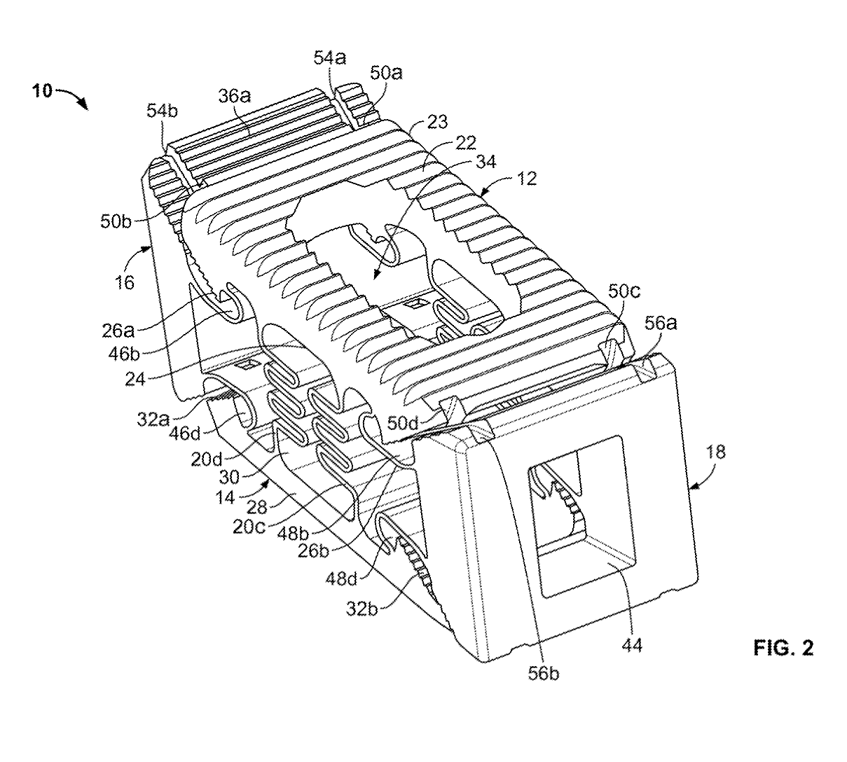

First wedge 16 can further include an angled, bulleted, or rounded exterior surface for aiding in insertion of implant 10 between adjacent vertebrae. In the variation shown in FIGS. 1-6, first wedge 16 includes rounded exterior surfaces 40a-d, which provides the bulleted nature of the exterior to the element. Angled surfaces can also be employed to achieve essentially the same functionality. First wedge 16 can also include a first wedge aperture 42 (shown in FIG. 1) formed therethrough and second wedge 18 can include a second wedge aperture 44 (shown in FIG. 2) formed therethrough. Both of these additional elements can be provided for use during expansion of implant 10.

Struts 20a-d can be deformable so as to allow for the expansion of implant 10 upon the movement of first and second members 12 and 14 away from one another. There are many different designs for such deformable struts that can be employed. For example, as is shown in FIG. 16, struts 20a-d are of an s-curve shape which facilitate easy compression and expansion. Struts 20a-d can be designed so that they apply tension to first and second members 12 and 14 during and after expansion of implant 10. This encourages even deployment of the device. More particularly, each of struts 20a-20d incorporates a specific structure designed to aid in the movement in first and second members 12 and 14 away from one another. As is shown in FIG. 5, each of the struts (of which only struts 20a and 20b are shown in FIG. 5) includes at least one curved section 102, which is designed to be thicker than at least one middle section 104, such that the curved section 102 will deform subsequent to the deformation of middle section 104. Each strut can include at least one end section 106 that is joined to one of end plates 12 and 14. This end section 106 can be thicker, such that there is no deformation at this point at anytime during the entire expansion sequence. The specific configuration of struts 20a-d facilitates the even deployment of implant 10 by specifically providing a structure that allows for a predetermined and consistent expansion sequence.

First and second wedges 16 and 18 are each respectively attached to both first and second members 12 and 14. As is shown in FIGS. 1-6, first wedge 16 is attached to first member 12 through the use of tethers 46a and 46b, and to second member 14 through the use of tethers 46c and 46d. Likewise, second wedge 18 is attached to first member 12 through the use of tethers 48a and 48b, and to second member 14 through the use of tethers 48c and 48d. Of course, any number of tethers can be utilized in connecting the wedges to the first and second members. Tethers 46a-d and 48a-d can be deformable so as to allow the movement of first and second wedges 16 and 18 with respect to first and second members 12 and 14. As is shown in the figures, the tethers can employ a shape that allows them to deform in a proper fashion upon movement of first and second wedges 16 and 18 with respect to first and second members 12 and 14. Like struts 20a-d, tethers 46a-d and 48a-d incorporate a structure specifically designed to allow for an even and consistent deployment of implant 10. Specifically, each tether includes an end section 110 (shown in connection with the illustration of tethers 46a, 46c, 48a, and 48c in FIG. 5) at the connection between the tether and one of first or second members 12 or 14, which is thicker than other areas of the tether to limit deformation. In addition, this section 110 is shaped in the manner shown in order to force a thinner curved tether section 112 to deform toward either the first or second member during the initial expansion of implant 10. This specific geometry results in the tether's initial movement to be a collapsing motion at section 110. Furthermore, each of tethers 46a-d and 48a-d include a connection section 114 at the connection between the tether and one of first or second wedges 16 or 18. This section, like section 110, is thicker than section 112 to limit the amount of deformation at the coupling of the tether and the wedge. The final expanded state of implant 10 is shown in FIG. 6, which illustrates the final position of the tethers.

In order to be suitable for implantation into the human body, all of the elements of implant 10 can be biocompatible. For example, in a variation, each of the components of implant 10 is constructed of a metal, such as titanium (commercially pure grade 2). However, other biocompatible materials can be utilized, like other titaniums, PEEK, titanium/PEEK composites, nitinol, bioresorbables, and the like. Depending upon the material utilized, certain of the components can be formed integral with or separately from one another. For example, struts 20a-d, in certain variations, can be formed integral with first and second members 12 and 14. In other variations, struts 20a-d and first and second members 12 and 14 can be formed separately and constructed together in accordance with normal practices. For instance, these portions could be welded or otherwise fused together.

Implant 10 also can include certain elements which cooperate to substantially prevent torsional movement of the first and second wedges 16 and 18 with respect to first and second members 12 and 14. Of course, such elements are not required for proper operation of the device. As is shown in FIGS. 1-6, first and second members 12 and 14 are provided with elongate protuberances (50a-d and 52a-d, respectively). These protuberances can extend somewhat below the angled interior surfaces of first and second members 12 and 14, respectively. First and second wedges 16 and 18, on the other hand, each include four channels for cooperation with the protuberances. Specifically, first wedge includes channels 54a-d and second wedge includes channels 56a-d.

The cooperation between the above-discussed protuberances and channels is such that movement of wedges 16 and 18 with respect to each other and first and second members 12 and 14 is not inhibited (i.e., the wedges can move in similar directions as depicted by arrows A and B of FIG. 5). However, any torsional or rotational movement of the wedges with respect to the first and second members is prevented. In other words, first and second wedges 16 and 18 are prevented from going off track. This is an important feature in ensuring a consistent operation of implant 10.

In operation, movement of first wedge 16 in the direction of arrow A (FIG. 5) and movement of second wedge 18 in the direction of arrow B (also Figure “S), causes first and second members 12 and 14 to move away from one another. In other words, movement of first and second wedges 16 and 18 towards one another causes the expansion of implant 10. First wedge aperture 42 can be threaded.

The deformable nature of tethers 46a-d and 48a-d allows them to follow along with first and second wedges 16 and 18 during their movement towards one another. So, at all times the wedges are connected to first and second members 12 and 14, thereby preventing them from becoming dislodged from implant 10. This is an important safety feature of the implant. Furthermore, the above-discussed teeth located on the first and second angled interior surfaces and the angled wedge surfaces allows for the movement of first and second wedges 16 and 18 in the direction of arrows A and B, respectively, but prevents opposite movement of the components. In other words, the different cooperating teeth (i.e., 27a and 37a, 27b and 39a, 33a and 37b, and 33b and 39b) are designed so as to allow the first movement, but prevent the second, opposite movement. Many different teeth designs can be employed in order to achieve this functionality.

Upon movement of first and second wedges 16 and 18 towards one another, first and second members 12 and 14 expand, which can act to both distract the vertebral space and also dig projections 23 and 29 of the vertebral contact surfaces 22 and 28 into the vertebral end plates of the vertebra they are in contact with. As is mentioned above, the different cooperating teeth (i.e., 27a and 37a, 27b and 39a, 33a and 37b, and 33b and 39b) allow for the expansion of implant 10, but prevent its contraction. Thus, once expanded, implant 10 remains in such a state without the addition of any further components. Nonetheless, one or more locking components could be utilized to ensure that implant 10 remains in the expanded state.

It is to be understood that the above brief discussion of the surgical procedure is merely exemplary, and more, less, or different steps can be performed. Moreover, one or more implant 10 can be inserted and deployed between adjacent vertebrae. Depending upon the overall size of the implant (which can widely vary), more than one implant can be required in order to properly support the disc space. With the implant(s) in place and deployed, the disc space can be restored to at or near its original height. Bone growth can occur through apertures 34 and 36 of the first and second members 12 and 14, respectively. First and second wedges 12 and 14 can include similar apertures or voids which ensure an open passage through implant 10 upon full expansion. In the expanded state, the interior of implant 10 can be packed with bone morphonogenic proteins or other bone growth inducing substances in order to encourage this bone growth from one adjacent vertebra to the other.

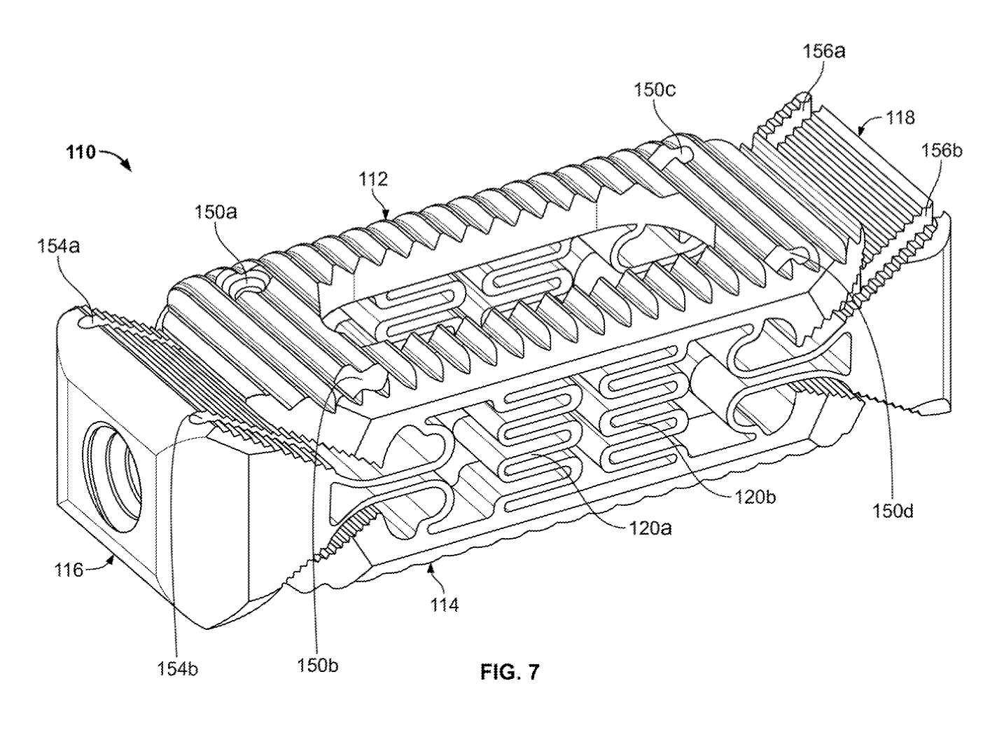

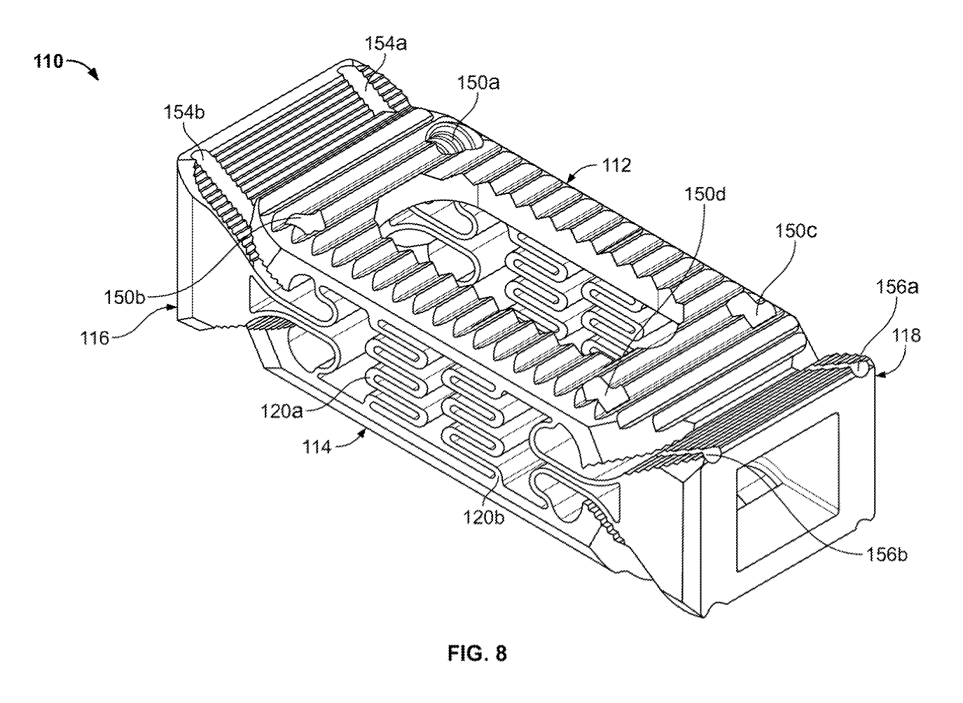

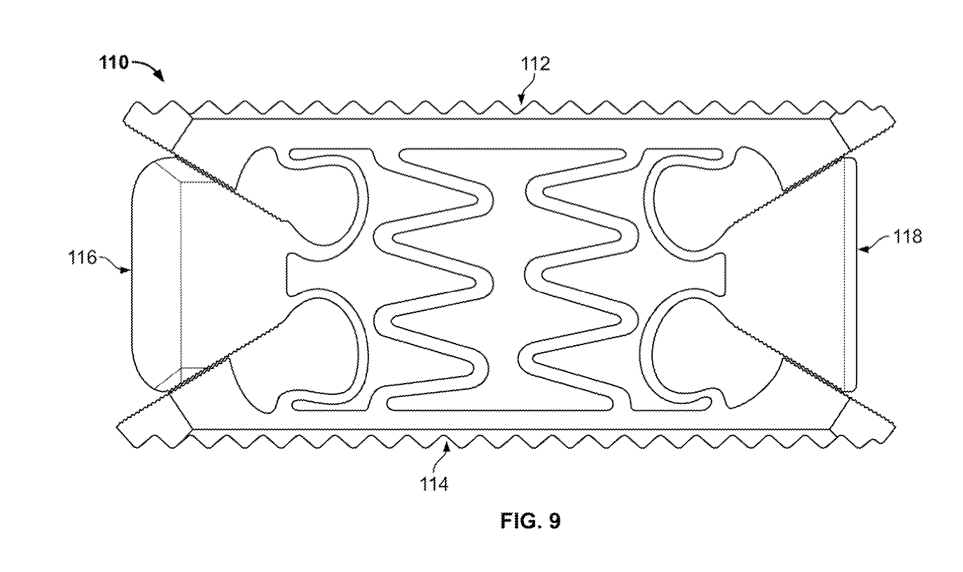

FIGS. 7-9 depict a second variation implant 110. Essentially, implant 110 is substantially similar to implant 10 save for the inclusion of different torsion inhibiting elements. Because of the similarity of implant 110 with implant 10, similar or identical elements will be referred to with like reference numerals within the 100-series of numbers. For example, implant 110 includes first and second members 112 and 114 which are expandable upon movement of first and second wedges 114 and 116 towards one another. However, in the variation shown in FIGS. 7-9, first and second members 112 and 114 are provided with apertures (150a-d and 152a-d, respectively) which are capable of receiving protuberances (not shown). For example, these apertures can receive pins, screws, or plugs which extend somewhat below the angled interior surfaces of first and second members 112 and 114, respectively. First and second wedges 116 and 118, on the other hand, each include four channels for cooperation with the protuberances. Specifically, first wedge includes channels 154a-d and second wedge includes channels 156a-d.

The cooperation between the protuberances and channels is like that that similar elements of implant 10 such that movement of wedges 116 and 118 with respect to each other and first and second members 112 and 114 is not inhibited. However, any torsional or rotational movement of the wedges with respect to the first and second members is prevented. In other words, first and second wedges 116 and 118 are prevented from going off track.

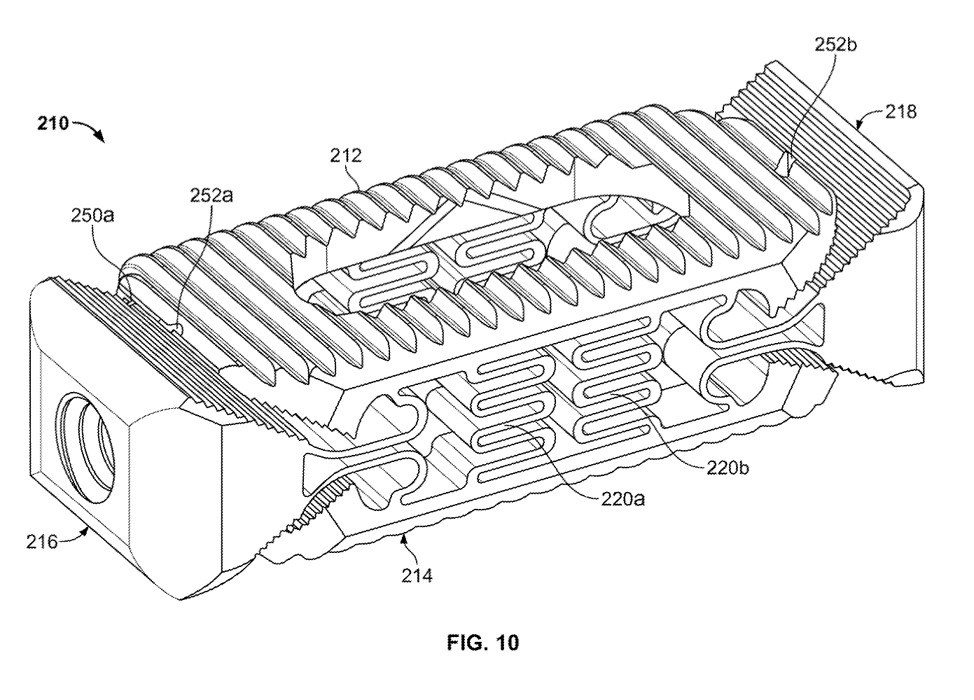

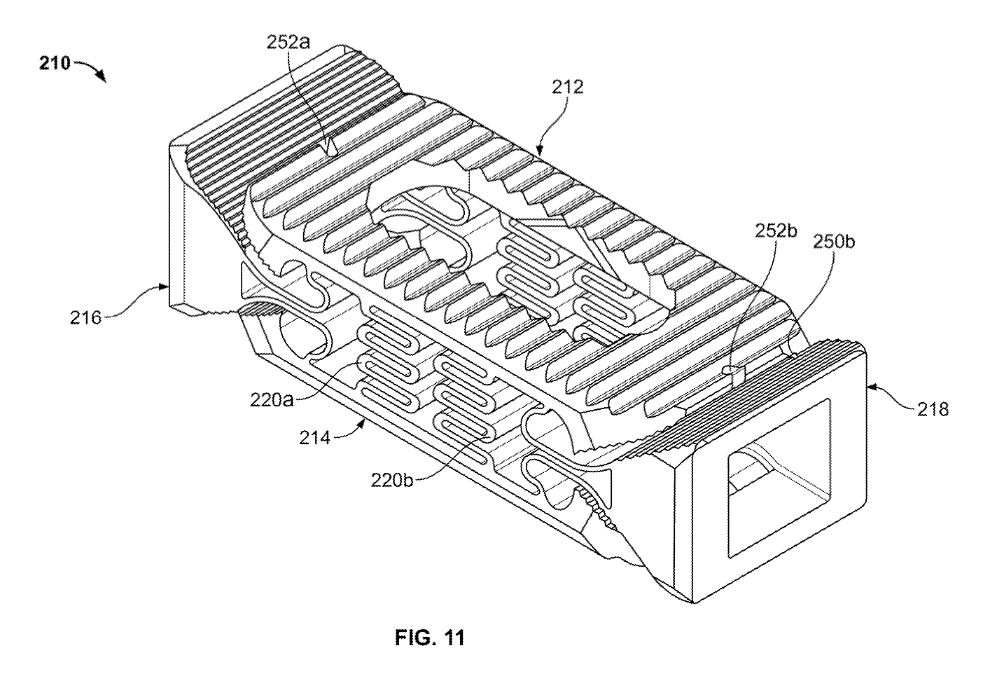

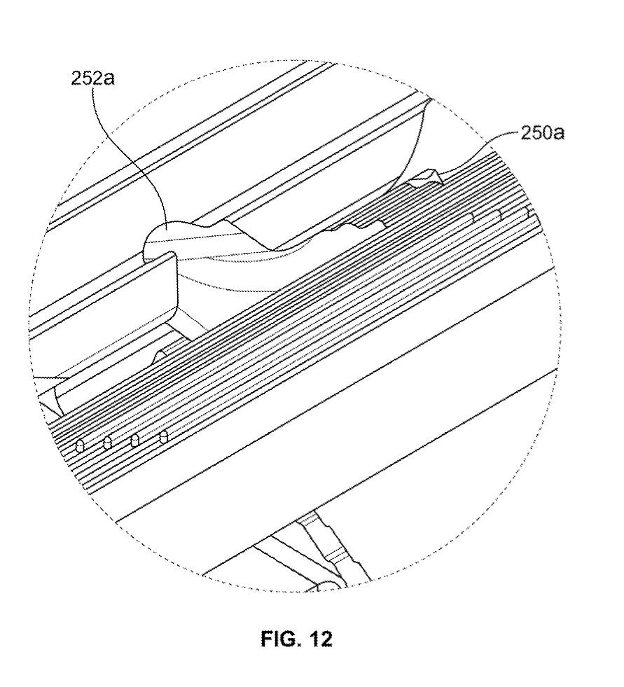

FIGS. 10-12 depict yet another variation implant 210. Like, implant 110, implant 210 is similar to implant 10, save for the inclusion of different torsion inhibiting elements. Once again, like elements in implant 210 will be referred to within the 200-series of numbers. Instead of including a series of channels and protuberances, the torsion inhibiting elements of implant 210 include a tongue and groove cooperation between its first and second members 212 and 214 and its first and second wedges 216 and 218. Specifically, first wedge 216 is provided with a first tongue 250a for cooperation with a first groove 252a of the first member, and a second tongue 250b for cooperation with a second groove 252b of the first member. Likewise, second wedge 218 is provided with a first tongue 250c for cooperation with a first groove 252c of the first member, and a second tongue 250d for cooperation with a second groove 252d of the second member. These elements cooperate in order to provide a nearly identical function to that of the torsion inhibiting elements discussed above in connection with implant 110. Each of the above discussed torsion inhibiting elements can vary. For instance, the specific shapes of the elements can widely vary. The inclusion of certain elements on certain components can be swapped. For example, implant 210 can include wedges employing grooves and first and second members employing tongues.

Although the disclosure has been described with reference to particular variations, it is to be understood that these variations are merely illustrative of the principles and applications of the disclosure. It is therefore to be understood that numerous modifications can be made to the illustrative variations and that other arrangements can be devised without departing from the spirit and scope of the present invention as defined by any claims presented.

We claim:

1. An orthopedic implant device comprising:
a first plate facing in a first direction;
a second plate facing in a second direction; and
a first wedge, wherein the first wedge is tethered to the first plate.

2. The device of claim 1, wherein the first wedge is positioned between the first plate and the second plate.

3. The device of claim 1, wherein the first wedge is tethered to the second plate.

4. The device of claim 1, wherein the first direction is opposite to the second direction, and where in the first plate is configured to move in the first direction with respect to the second plate.

5. The device of claim 1, wherein the first edge is configured to spread the first plate away from the second plate when the first wedge is moved toward the center of the orthopedic implant.

6. The device of claim 1, further comprising a second wedge between the first plate and the second plate.

7. The device of claim 6, wherein the second wedge is tethered to the first plate.

8. The device of claim 7, second wedge is tethered to the second plate.

9. The device of claim 1, further comprising a first strut between the first plate and the second plate.

10. The device of claim 9, wherein the first strut is in tension between the first plate and the second plate.

11. The device of claim 1, further comprising a second strut between the first plate and the second plate.

12. A method for providing orthopedic support at a target site comprising:
implanting an expandable device to the target site, wherein the device comprises a first plate, a second plate, a first wedge between the first plate and the second plate, and wherein the first wedge is tethered to the first wedge; and
expanding the first plate away from the second plate, wherein expanding comprises moving the first wedge toward the center of the expandable device.

13. The method of claim 12, further comprising tethering the first wedge to the first plate.

14. The method of claim 13, wherein the expandable device comprises a tether attaching the first wedge to the first plate, and wherein the tether tethers the first wedge to the first plate.

15. The method of claim 12, wherein the expandable device further comprises a second wedge between the first plate and the second plate, and wherein expanding the first plate away from the second plate further comprises moving the second wedge toward the center of the expandable device.

16. The method of claim 15, further comprising tethering the second wedge to the first plate.

17. The method of claim 16, wherein the expandable device comprises a tether attaching the second wedge to the first plate, and wherein the tether tethers the second wedge to the first plate.

18. The method of claim 12, further comprising tensioning the first plate to the second plate.

19. The method of claim 18, wherein the tensioning comprises delivering a tensioning force, and wherein the expandable device comprises a first strut integral with the first plate and the second plate, and wherein the first strut delivers the tensioning force between the first plate and the second plate.

20. The method of claim 19, wherein the expandable device comprises a second strut integral with the first plate and the second plate, and wherein the first strut and the second strut deliver the tensioning force between the first plate and the second plate.
